(19) United States
(12) Patent Application Publication
(10) Pub. No.: US 2021/0000729 A1
(43) Pub. Date: Jan. 7, 2021

(54) METHOD OF MANUFACTURING COMPOSITION MATERIAL FOR SKIN MOISTURIZING CONTAINING VEHICLE HAVING MULTI-LAYER GLOBULE

(71) Applicants: VENN SKINCARE, INC., Los Angeles, CA (US); VENN SKINCARE KOREA LLC, Seoul (KR)

(72) Inventor: Jae-Choel MUN, Cheongju-si, Chungcheongbuk-do (KR)

(21) Appl. No.: 16/979,525

(22) PCT Filed: Mar. 16, 2018
(86) PCT No.: PCT/KR2018/003116

§ 371 Date: Sep. 10, 2020

(51) Int. Cl. A61K 8/64; A61K 8/55; A61K 8/06; A61Q 19/00

(52) U.S. Cl. CPC A61K 8/64; A61K 8/553; A61Q 19/007; A61K 8/064; A61K 8/062

(57) ABSTRACT

Disclosed is a method of manufacturing a composition material for skin moisturizing containing a vehicle having a multi-layer globule. The method includes manufacturing a hydrogenated lecithin dispersion solution by adding hydrogenated lecithin to purified water at 70 to 90° C. with agitation to thus perform dispersion, manufacturing a pre-emulsion base by adding a saturated fatty alcohol having 12 to 22 carbon atoms to the hydrogenated lecithin dispersion solution at 70 to 90° C. with agitation to thus perform dispersion, manufacturing a composition material base for skin moisturizing by adding the pre-emulsion base and water-soluble polypeptide to the purified water at 70 to 90° C. with agitation to thus perform dispersion, and manufacturing a multi-layer globule for skin moisturizing including phospholipid bilayers by adding oil to the composition material base for skin moisturizing at 70 to 90° C. with agitation to thus perform dispersion.

### TECHNICAL FIELD

The present invention relates to a method of manufacturing a composition material for skin moisturizing containing a vehicle having a multi-layer globule. More particularly, the present invention relates to the use of a pre-emulsion base and a multi-layer globule for skin moisturizing including phospholipid bilayers in a composition material for skin moisturizing containing a vehicle having a multi-layer globule. The pre-emulsion base is manufactured using hydrogenated lecithin and saturated fatty alcohol having 12 to 22 carbon atoms. After water-soluble polypeptide is added to the pre-emulsion base to thus perform stabilization, oil is added thereto, thereby manufacturing the multi-layer globule for skin moisturizing including the phospholipid bilayers.

### BACKGROUND ART

The skin performs various functions that affect the metabolism of the body, and serves to protect internal organs from external factors breaking the homeostasis of the body and to protect life. The main functions of the skin are: 1) to maintain the balance of water in the skin, 2) to protect the body as live outermost part of the body that protects the internal organs. 3) to act as a barrier that prevents the loss of water and heat, and 4) to perform self-regeneration that maintains homeostasis.

There are two layers that are important for maintaining the homeostasis of water in the skin.

The first is the stratum corneum of the skin. In the stratum corneum, corneocytes and intercorneocyte lipids form a multi-lamellar layer structure, thus blocking the outflow of water from the body, acting as a defense wall that prevents the entry of harmful substances into the body, acting as a bonier to obstruct the delivery of dermatologically active substances from the stratum corneum of the skin epidermis to the dermis layer, and playing a crucial role in significantly lowering the permeability of dermatologically active substances. The constitution thereof includes 59% keratin, 31 to 38% natural moisturizing factor (NMF), and 11% intercellular lipids. Particularly, the intercellular lipids include ceramide, cholesterol, and fatty acid.

The second is the stratum granulosum of the skin epidermis. The stratum granulosum contains substances such as glycerol, byaluronic acid, and collagen, which help to prevent water from escaping to the outside. Further, filaggrin, which is a protein playing an important role in skin barriers, is present in the stratum granulosum to thus maintain water homeostasis in the skin in the case of lack of water in the skin. This protein serves to decompose free fatty acids present in the skin into water so as to maintain water homeostasis in the skin in the case of lack of water in the skin.

The skin consists of at least about 70% water, and it is absolutely necessary to maintain adequate water. If water retention is impaired, it can lead to serious symptoms, such as dryness of the skin and the formation of wrinkles.

The most ideal system for skin moisturizing may be a three-dimensional moisturizing system that satisfies all of the steps of 1) supplying water from the inside (stratum granulosum) of the skin to thus maintain water homeostasis in the skin, 2) maintaining phospholipid layers present in the outer skin layer (stratum corneum) in a healthy state, and 3) forming a skin-moisturizing membrane so that water is not lost from the skin epidermis to the atmosphere.

However, polyhydric alcohols, water-soluble polymers, and organic acid salts are currently used as skin moisturizers, anti a method of increasing water retention in the stratum corneum using an occlusive ingredient, which is oily and which prevents water from evaporating from the surface of the skin, and a humectant having a property of absorbing water as moisturizers, is widely applied.

Examples of the humectant include substances such as glycerin, propylene glycol, 1,3-butylene glycol, and polyethylene glycol as polyols. As the occlusive ingredient, lipid ingredients such as ceramide or essential fatty acids and lipid complexes are used. However, they have drawbacks in that since the moisturizing effect is temporary, the long-term skin-moisturizing effect is rot continuously maintained, making it difficult to apply the same to various formulations.

Further, there ate problems in that water supply from the inside of the skin is insufficient and in that the delivery of the occlusive ingredient, which is oily and which prevents water from evaporating from the surface of the skin, and of the humectant having the property of absorbing water to the inside of the skin is insufficient, and thus the skin-moisturizing effect is not effective.

Korean Patent No. 10-1805395 discloses a cosmetic composition material for skin moisturizing. In the cosmetic composition material for skin moisturizing, ceramide, glycerin, and butylene glycol are mixed at a predetermined content ratio for use. whereby a moisture-protection membrane is efficiently formed on the skin so that an excellent skin-moisturizing effect is realized. However, there still remain problems in that the long-term skin-moisturizing effect is not continuously maintained because of the temporary moisturizing effect thereof and in that water supply from the inside of the skin is insufficient and the delivery of the occlusive ingredient and the humectant to the inside of the skin is insufficient, and accordingly, the skin-moisturizing effect is not effective.

### Document of Related Art

[Patent Document]

Patent Document 0001) Korean Patent No. 10-1805395 (Announcement date: Jan. 10, 2018)

### DISCLOSURE OF INVENTION

### Technical Problem

Accordingly, the present invention las been made keeping in mind the above problems occurring in the prior art, and an object of the present invention is to provide a method of manufacturing a composition material for skin moisturizing containing a vehicle having a multi-layer globule. The flexible liposome structure thereof, which resembles water balloons, can smoothly pass through the narrow gaps in the skin, unlike other micelle structures, by manufacturing a composition material for skin moisturizing containing a vehicle having a multi-layer globule having a multi layer structure in which a hydrophilic layer and a hydrophobic layer alternate with each other using a stable multi-layer globule (liposome structure) including phospholipid bilayers. Therefore, water-soluble polypeptide is stably delivered to the stratum granulosum of the skin.

Another object of the present invention is to provide a method of manufacturing a composition material for skin moisturizing containing a vehicle which has a multi-layer globule and which stably delivers a water-soluble natural moisturizing factor and a water-soluble or oil-soluble moisturizing-membrane-forming agent to each layer of the skin while passing through various tissues of the skin, in which a hydrophilic layer and a hydrophobic layer alternate with each other, using a multi-layer structure which includes phospholipid bilayers and in which a hydrophilic layer and a hydrophobic layer alternate with each other.

Yet another object of the present invention is to provide a method of manufacturing a composition material for skin moisturizing, containing a vehicle having a multi-layer globule, by which a stable water-soluble polypeptide vehicle is easily manufactured without the need for complicated processes or expensive apparatuses.

Still another object of the present indention is to provide a method of manufacturing a composition material for skin moisturizing containing a vehicle having a stable multi-layer globule, which is not affected by surrounding environments such as temperature or humidity and in which the multi-layer globule including phospholipid bilayers is not destroyed despite the passage of time and is maintained while passing through various tissues of the skin, in which a hydrophilic layer and a hydrophobic layer alternate with each other.

### Solution to Problem

In order to accomplish the above objects,

the present invention provides a method of manufacturing a composition material for skin moisturizing containing a vehicle having a multi-layer globule, the method including manufacturing a hydrogenated lecithin dispersion solution by adding hydrogenated lecithin to purified water at 70 to 90° C. with agitation to thus perform dispersion, manufacturing a pre-emulsion base by adding saturated fatty alcohol having 12 to 22 carbon atoms to the hydrogenated lecithin dispersion solution at 70 to 90° C. with agitation to thus perform dispersion, manufacturing a composition material base for skin moisturizing by adding the pre-emulsion base and a water-soluble polypeptide to the purified water at 70 to 90° C. with agitation to thus perform dispersion, and manufacturing a multi-layer globule for skin moisturizing including phospholipid bilayers by adding oil to the composition material base for skin moisturizing at 70 to 90° C. with agitation to thus perform dispersion.

### Advantageous Effects of Invention

A method of manufacturing a composition material for skin moisturizing containing a vehicle having a multi-layer globule which is the present invention, has the following effects.

First, there is provided a composition material for skin moisturizing containing a vehicle having a multi-layer globule, in which the flexible and stable liposome structure thereof, which resembles water balloons, can smoothly pass through the narrow gaps in the skin, unlike other micelle structures, by manufacturing a composition material for skin moisturizing containing a vehicle having a multi-layer globule having a multi-layer structure in which a hydrophilic layer and a hydrophobic layer alternate with each other using a multi-layer globule (liposome structure) including phospholipid bilayers. Therefore, a water-soluble polypeptide (particularly, RH-polypeptide-64 (aquaporin)) is stably delivered to the stratum granulosum of the skin without damaging the vehicle.

Second, the present invention provides a composition material for skin moisturizing containing a vehicle having a multi-layer globule, the vehicle having a multi-layer structure including phospholipid bilayers in which a hydrophilic layer and a hydrophobic layer alternate with each other. The vehicle stably delivers a water-soluble natural moisturizing factor and a water-soluble or oil-soluble moisturizing-membrane-forming agent to each layer of the skin while easily passing through various tissues of the skin in which a hydrophilic layer and a hydrophobic layer alternate with each other.

Third, in the present invention, a composition material for skin moisturizing containing a vehicle having a stable multi-layer globule including at least two phospholipid bilayers may be easily manufactured without the need for complicated processes or expensive apparatuses, whereby economical effects are secured.

Fourth, since the present invention is not affected by surrounding environments, such as temperature or humidity, and a multi-layer globule including phospholipid bilayers is stably maintained despite the passage of time, a water-soluble or oil-soluble natural moisturizing factor and an oil-soluble moisturizing-membrane-forming agent are stably delivered to each layer of the skin while passing through various tissues of the skin, in which a hydrophilic layer and a hydrophobic layer alternate with each other.

### BRIEF DESCRIPTION OF DRAWINGS

FIG. 1A is a first mimetic view showing activation of an internal water supply system;

FIG. 1B is a second mimetic view showing a process for activating the internal water supply system;

FIG. 1C is a mimetic view showing the structure of the water channel of aquaporin in the internal water supply system;

FIG. 2A is a view showing the reversible micelle structure of a hydrogenated lecithin dispersion solution manufactured by adding hydrogenated lecithin to purified water at 70 to 90° C. with agitation to thus perform dispersion;

FIG. 2B is a view showing the cross-sectional structure of a pre-emulsion base manufactured by adding saturated fatty alcohol having 12 to 22 carbon atoms to the hydrogenated lecithin dispersion solution at 70 to with agitation to thus perform dispersion;

FIG. 2C is a view showing the cross-sectional structure of a composition material base for skin moisturizing, manufactured by adding the pre-emulsion base and a water-soluble polypeptide, which is a water-soluble substance, to the purified water at 70 to 90° C. with agitation to thus perform dispersion;

FIG. 2D is a view showing the cross-sectional structure of a multi-layer globule for skin moisturizing including phospholipid bilayers manufactured by adding oil to the composition material base for skin moisturizing with agitation to thus perform dispersion;

FIG. 3 is a picture of a composition material for skin moisturizing containing the vehicle having the multi-layer globule, which is the present invention, taken at a magnification of 400 times using a polarizing microscope;

FIG. 4 is an ATR-FTIR image showing the hydration levels of the skin (control), which is not treated with the composition material for skin moisturizing containing the vehicle having the multi-layer globule that is the present invention, and the skin (VENNARC-001), which is treated with the composition material for skin moisturizing containing the vehicle having the multi-layer globule, using a peak area ratio of water to amide II cm⁻¹; and

FIG. 5 is a comparative graph showing the hydration levels of the skin (control), which is not treated with the composition material for skin moisturizing containing the vehicle having the multi-layer globule that is the present invention, and the skin (VENNARC-001), which is treated with the composition material for skin moisturizing containing the vehicle having the multi-layer globule, over time.

### BEST MODE FOR CARRYING OUT THE INVENTION

It is to be understood that the terms or words used in the present specification and claims are not to be construed in a conventional or dictionary sense and that the inventor can properly define the concept of a term to describe their invention in the best possible way. Accordingly, the present invention should be construed as having a meaning and concept consistent with the technical idea of the present invention. Therefore, the embodiments described in the specification of the present invention and the constitutions shown in the drawings are merely the most preferred embodiments of the present invention, and do not represent the entire technical scope of the present invention. It should be understood that various equivalents and modifications that may be substituted for these at the time of filing of the present invention are possible or may be present.

Before describing the present invention with reference to the following Examples, it should be noted that what is not necessary in order to disclose the gist of the present invention, that is, a known constitution that can be obviously added by a person skilled in the art, is not shown in the drawings and not specifically described.

The most desirable system for skin moisturizing proposed by the present invention may be a system that satisfies all of the steps of 1) supplying water from the inside (stratum granulosum) of the skin to this maintain water homeostasis in the skin, 2) maintaining phospholipid layers present in the outer skin layer (stratum corneum) in a healthy state, and 3) forming a skin-moisturizing membrane so that water is not lost from the skin epidermis to the atmosphere.

The skin-moisturizing system proposed by the present invention is as follows.

1) Internal Water Supply System [Internal Water Supply System by Aquaporin]

In order to maintain water homeostasis in the skin, when the water content in the skin is insufficient, the aquaporin ingredient, which is known to activate filaggrin, may be delivered from the outside to the stratum granulosum in the skin so that the filaggrin is activated to thus activate the system for supplying water from the inside of the skin, whereby the dry ness of the inside of the skin is reduced, thus overcoming a skin pulling-phenomenon, which occurs when the skin is dry, and a skin-drying phenomenon. In the present invention, a water-soluble polypeptide (aquaporin) ingredient known to activate filaggrin is stably delivered from the outside to the stratum granulosum in the skin using a multi-layer globule for skin moisturizing including phospholipid bilayers, which activates the filaggrin, thus activating the system for supplying water from the inside of the skin.

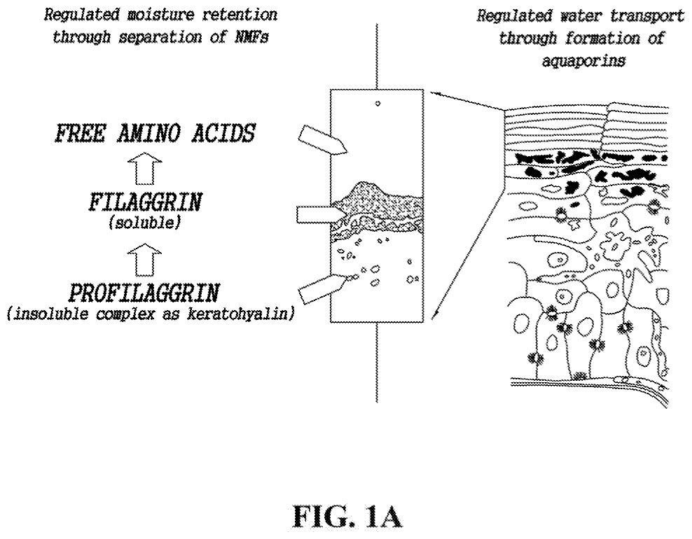

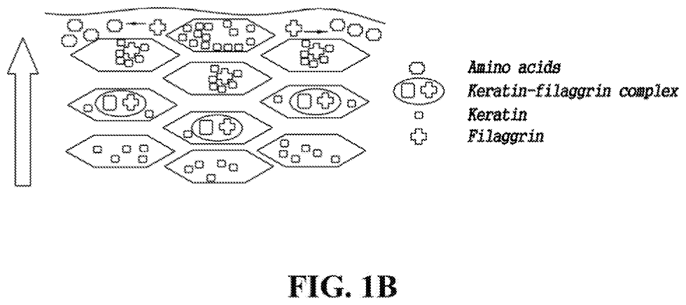

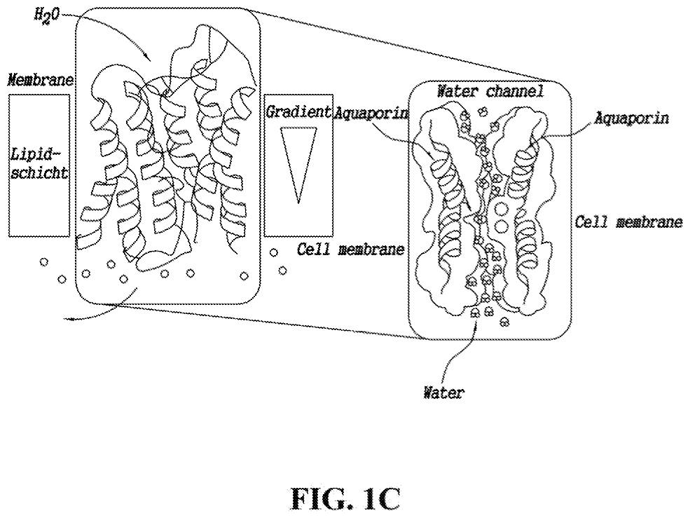

FIG. 1A is a first mimetic view showing activation of an internal water supply system. FIG. 1B is a mimetic view showing a process for activating the internal water supply system, and FIG. 1C is a mimetic view showing the structure of the water channel of aquaporin in the internal water supply system.

According to the above drawings, when the skin is dried, the following internal water supply system [internal water supply system by aquaporin] operates in the skin in order to maintain the homeostasis of water.

Step 1. Keratin is granulated in the cells of the epidermis.

Step 2. Also, filaggrin is produced to form a complex with keratin. This complex prevents the filaggrin from being destroyed [Profilaggrin: Keratin-filaggrin complex].

Step 3. When this complex moves from the stratum granulosum to the stratum corneum, a specific enzyme destroys bonds inside the keratin-filaggrin complex.

Step 4. In addition, keratin in the corneocyte of the filaggrin in the stratum corneum is arranged at the outer side thereof.

Step 5. The thus arranged filaggrin is decomposed into free amino acids, which are a natural moisturizing factor, using a specific enzyme.

Step 6. This free amino acid flows into a TCA cycle to thus supply sufficient water to the inside of the skin cells by generating the sufficient water. When the water homeostasis in the skin is restored, the process of steps 1 to 6 is stopped, and no more water is supplied to the skin.

Step 7. The sufficient water generated in the cell moves to the stratum corneum through the fine water channel formed by the peptide ingredient called aquaporin between the cell membranes of the skin, thus maintaining water homeostasis in the skin.

In the past, it was thought that water entered the skin cells through diffusion. However, multiple studies have confirmed that the rale of movement of water into cells is 10 to 100 limes faster than the rate of diffusion. For this reason, many scientists have predicted that there is another unknown route for water movement. As a result, it was confirmed in 1997 that there is a specific protein that promotes the influx of water, and this protein was named aquaporin. Aquaporin is a protein that forms a channel through which water can move in the cell wall It was confirmed using an electron microscope that water is transported between the cells through a channel including four aquaporin proteins. Aquaporin forms nano-sized channels and is selectively permeable only to water molecules, and, with respect to its transport capacity, it was confirmed that 3 billion water molecules are transported per second per aquaporin channel. Therefore, in order to activate the internal water supply system [internal water supply system by aquaporin], as in the present invention, a water-soluble polypeptide (aquaporin) ingredient known to activate filaggrin may be stably delivered from the outside to the stratum granulosum in the skin using a multi-layer globule for skin moisturizing including phospholipid bilayers, thus activating the filaggrin. Thereby, a system for supplying water from the inside of the skin is operated, and sufficient water generated in cells moves to the stratum corneum through the fine water channel formed by the aquaporin between the cell membrane* of the skin, thus maintaining water homeostasis in the skin.

2) Method of Supplying Water to Skin from Outside [External Water Supply Method by NMF]

Another method of supplying water to the skin to prevent the skin from being dried is to supply a natural moisturizing factor to the surface of the skin so that the water state of the skin surface becomes a saturated state, thus maintaining the water of the skin. This is a method in which, by applying the cosmetic composition material containing hyaluronic acid, amino acid, and collagen ingredients, which are natural moisturizing factors present in the skin, polymer ingredients, and wax ingredients mixed with each other on the surface of the skin, the natural moisturizing factors are not dried anti the loss of water contained in natural moisturizing factors is minimized due to the polymer components or wax components that are used, whereby the skin is not dried and water is maintained therein over a long period of time. In this case, in the present invention, the natural moisturizing factor ingredient may be stably delivered from the outside to the inside of the skin using the multi-fiver globule for skin moisturizing including the phospholipid bilayers, whereby the natural moisturizing factor is efficiently and stably embodied.

3) Prevention of Water Loss from Skin [Protection Against Water Loss from Skin Surface]

The internal water supply method using filaggrin and aquaporin and the water supply method from the outside of the skin using the natural moisturizing factor are important for maintaining the water of the skin, but fundamentally important is to prevent the water in the skin from being lost. The most common cause of water escaping from the skin to the air is damage to phospholipids constituting the membrane of skin cells. When the phospholipids surrounding the skin cells are damaged, the water present in the skin cells is taken away into the air and the skin is rapidly dried, causing the skin to suffer front diseases such as atopy. Therefore, in order to stably and ultimately secure the water homeostasis in the skin, a method of restoring or regenerating the phospholipid membrane of damaged skin cells is needed. In the present invention, an oil-soluble moisturizing-membrane-forming agent ingredient may be stably delivered from the outside to the inside of the skin using a multi-layer globule for skin moisturizing including phospholipid bilayers, thus restoring the damaged phospholipid.

As described above, simply applying the cosmetic composition material on the skin cannot fundamentally solve the problem of moisturizing, and the actions of all of 1), 2) and 3) need to be complexly performed in order to fundamentally and effectively solve the problem of skin moisturizing. Therefore, it is necessary to provide the multi-layer globule for skin moisturizing of the present invention, including phospholipid bilayers in which a hydrophilic layer and a hydrophobic layer alternate with each other. In general, water-soluble polypeptide (aquaporin) has a hydrophilic property and thus easily dissolves in water. Accordingly, it cannot pass through the stratum corneum and hydrophobic intercorneocyte lipids and cannot be delivered to the stratum granulosum in the skin. Further, a phospholipid ingredient is required in order to restore the damaged skin, but since the molecular weight of the phospholipid ingredient is very high, the phospholipid ingredient is not delivered into the skin. Therefore, since the ingredient is not delivered to the inside of the skin using a general water-in-oil type or oil-in-water type emulsification method, only the use of the multi-layer globule for skin moisturizing of the present invention, which is a special vehicle (delivery vehicle), can fundamentally solve the problem of skin moisturizing. In order to activate the water supply system in each layer of the skin, the applied vehicle (delivery vesicle) must include at least two phospholipic bilayers, and must have a layer structure in which hydrophilic and hydrophobic layers alternate with each other (hydrophilic-hydrophobic layer structure).

Human skin plays the true role thereof only when it maintains the homeostasis of water, and maintaining water homeostasis is a very important role thereof. The present invention provides a composition material that provides a three-dimensional water supply system for 1) activating filaggrin, which is a protein delivered to the inside of the skin using a multi-layer vehicle containing water-soluble polypeptide to thus play an important role in water production in the skin, thereby supplying water from the inside of the skin, 2) supplying water to the skin to thus maintain water thereof over a long period of lime, and 3) reducing water loss.

In order to activate the internal water supply system of the skin, the present invention employs an emulsification technique for manufacturing a multi-layer globule for skin moisturizing including phospholipid bilayers containing a water-soluble polypeptide, and basically provides a composition material for skin moisturizing containing a vehicle having a multi-layer globule, thus supplying water to the skin and controlling water loss of the skin.

The prevent invention is based on three basic functions.

1) Internal Water Supply System [Internal Water Supply System by Aquaporin]

An emulsification technique for manufacturing a multi-layer globule for skin moisturizing including phospholipid bilayers is applied to provide an emulsification technique for delivering a representative aquaporin ingredient of water-soluble peptide ingredients to the skin layer underneath the stratum corneum of the skin so that the filaggrin protein is activated to thus activate a system for supplying water from the inside of the skin. The aquaporin ingredient is also referred to as RH-polypeptide-64 (aquaporin). The essence of the present invention is to embody an internal water supply system [internal water supply system by aquaporin] for stably delivering a water-soluble polypeptide (especially aquaporin) to the skin layer underneath the stratum corneum of the skin using the composition material for skin moisturizing containing the vehicle having the multi-layer globule, thus activating the filaggrin protein. 2) The method of supplying water to the skin from the outside [external water supply method by NMF] and 3) prevention of water loss from skin [protection against water loss from skin surface], which are described in the following, are additionally and supplementary included for the purpose of concerted and three-dimensional skin moisturizing.

2) Method of Supplying Water to Skin from Outside [External Water Supply Method by NMF]

This is intended to provide a technique for emulsifying a composition material for skin moisturizing containing a vehicle having a multi-layer globule. When the composition material is applied on the skin, the composition material supplies water to the Surface and inside of the skin to thus perform hydration, thereby maintaining the water persistence in the skin over a Jong period of time. In this case, a water-soluble natural moisturizing factor is used, and has a water-soluble (hydrophilic) property. Accordingly, in the present invention, the water-soluble natural moisturizing factor may be added to the water-soluble polypeptide during the step of manufacturing the composition material base for skin moisturizing by adding the pre-emulsion base and the water-soluble polypeptide to purified water at 70 to 90° C. with agitation to thus perform dispersion, thereby manufacturing a composition material base for skin moisturizing containing a water-soluble natural moisturizing factor. Examples of the natural moisturizing factor include glycerin, panthenol (D form), betaine, sodium hyaluronate, amino acid, collagen, glycosaminoglycan, sodium chondroitin sulfate, or mucopolysaccharide, and may be used alone or in a mixture of one or more thereof. In the present invention, to the activation effect of the internal water supply system [internal water supply system by aquaporin] obtained by the delivery of the water-soluble peptide (aquaporin) ingredient to the skin layer underneath the stratum corneum of the skin using the composition material for skin moisturizing containing the vehicle having the multi-layer globule, the activation effect of the method of supplying water to the skin from outside [external water supply method by NMF] obtained by the delivery of the water-soluble natural moisturizing factor to the skin layer underneath the stratum corneum of the skin may be added, whereby a three-dimensional skin-moisturizing system in which water is supplied from the inside and the outside may be started.

3) Prevention of Water Loss from Skin [Protection Against Water Loss from Skin Surface]

In the three-dimensional skin-moisturizing system of the present invention, in which water is supplied from the inside and the outside, a composition material for skin moisturizing, which is applied on the surface of the skin so that a moisturizing membrane for preventing water loss from the skin is formed to thus reduce the water loss from the skin, is further required. In the present invention, the moisturizing-membrane-forming agent is included in the multi-layer globule for skin moisturizing including phospholipid bilayers, thereby solving the above-described problems. The moisturizing-membrane-forming agent may be broadly divided into water-soluble (hydrophilic) and oil-soluble (hydrophobic) moisturizing-membrane-forming agents. In the present invention, the water-soluble moisturizing-membrane-forming agent may be added to the water-soluble polypeptide during the step of manufacturing the composition material base for skin moisturizing by adding the pre-emulsion base and the water-soluble polypeptide to the purified water at 70 to 90° C. with agitation to thus perform dispersion, thereby manufacturing a composition material base for skin moisturizing containing a water-soluble moisturizing-membrane-forming agent. In the present invention, the oil-soluble moisturizing-membrane-forming agent may be added to oil during the step of manufacturing the multi-layer globule for skin moisturizing including the phospholipid bilayers by adding oil to the composition material base for skin moisturizing at 70 to 90° C. with agitation to thus perform dispersion, thereby manufacturing a composition material base for skin moisturizing containing an oil-soluble moisturizing-membrane-forming agent. Examples of the water-soluble moisturizing-membrane-forming agent include glyceride, sodium olivoyl glutamate, or xanthan gum, and may be used alone or in a mixture of one or more thereof. Examples of the oil-soluble moisturizing-membrane-forming agent include shea butter, beeswax, vegetable wax, mineral wax, animal wax, silicone, silicone gums, silicone emulsions, silicone elastomers, or vegetable butter, and may be used alone or in a mixture of one or more thereof. The constitution for preventing the loss of water supplied as described above may be added to thus complete a three-dimensional skin-moisturizing system for supplying water from the inside and tic outside of the skin and for preventing the loss of the water that is supplied.

In order to improve moisturizing of the skin, a vehicle capable of delivering the water-soluble polypeptide, the natural moisturizing factor, and the moisturizing-membrane-forming agent into the skin is required. The surfactant is the most important part in the manufacture of the vehicle. In the present invention, hydrogenated lecithin is used instead of lecithin because of the stability of the vehicle, and the pre-emulsion base is manufactured using hydrogenated lecithin and saturated fatty alcohol having 12 to 22 carbon atoms. As described above, a pre-treatment process (pre-mixing process) for manufacturing the pre-emulsion base is a key process to lower the high interfacial tension of the hydrogenated lecithin, thus improving compatibility with other substances and also improving the stability of the vehicle. The composition of the pre-emulsion base in this process includes 1) hydrogenated lecithin. 2) saturated fatty alcohol having 12 to 22 carbon atoms, and 3) water as a basic frame, and 1) ceramide, 2) sterol, 3) ethanol, and 4) fatty acid may be added in live manufacture of the pre-emulsion base.

As described above, the pre-treatment process (pre-mixing process) for manufacturing the pre-emulsion base in order to lower the high interfacial tension of the hydrogenated lecithin, thus improving the compatibility with other substances and the stability of the vehicle, includes manufacturing the hydrogenated lecithin dispersion solution by adding the hydrogenated lecithin to the purified water at 70 to 90° C. with agitation to thus perform dispersion and manufacturing the pre-emulsion base by adding saturated fatty alcohol having 12 to 22 carbon atoms to the hydrogenated lecithin dispersion solution at 70 to 90° C. with agitation to thus perform dispersion. The composition material base for skin moisturizing is manufactured by adding the pre-emulsion base and the water-soluble polypeptide to the purified water at 70 to 90° C. with agitation to thus perform dispersion. Thereafter, the multi-layer globule for skin moisturizing including the phospholipid bilayers is manufactured by adding oil to the composition material base for skin moisturizing at 70 to 90° C. with agitation to thus perform dispersion.

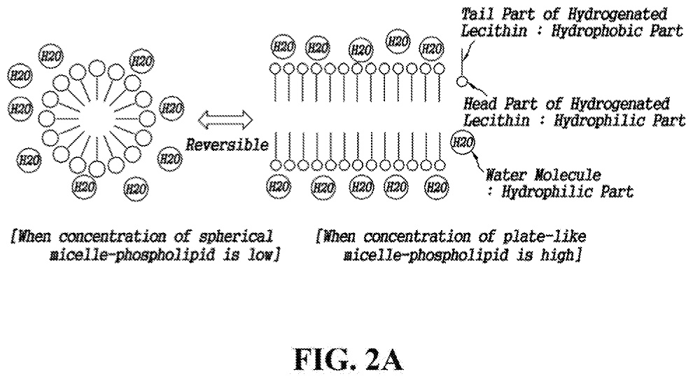

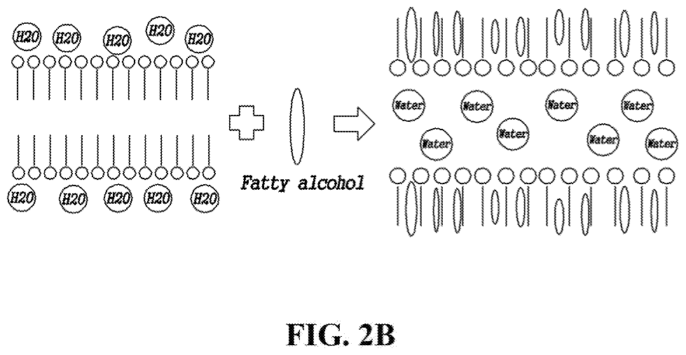

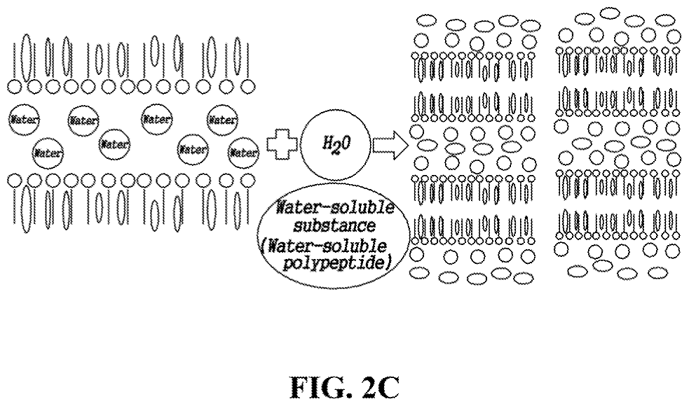

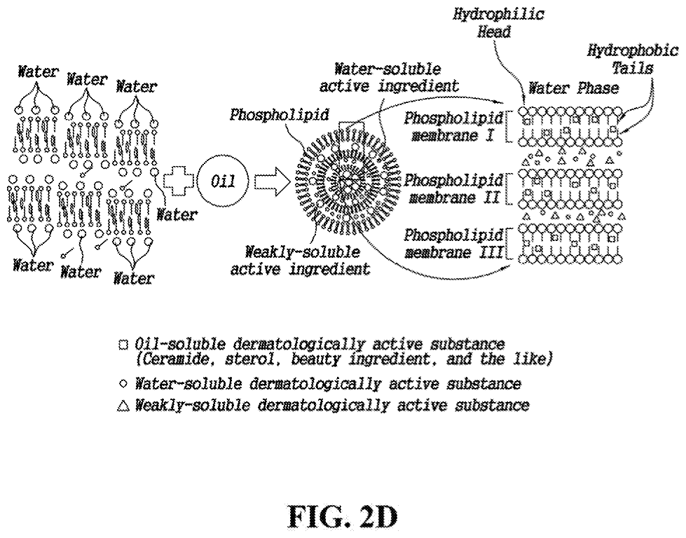

FIG. 2A is a view showing the reversible micelle structure of a hydrogenated lecithin dispersion solution manufactured by adding hydrogenated lecithin to purified water at 70 to 90° C. with agitation to thus perform dispersion.

According to FIG. 2A, after the purified water is healed to 70 to 90° C., the hydrogenated lecithin is slowly added thereto with agitation for 8 to 12 minutes (preferably for 10 minutes) under the condition of an ACl mixer (a general mixer) rotation speed of 800 to 1000 rpm, thus manufacturing a hydrogenated lecithin dispersion solution. The manufactured hydrogenated lecithin dispersion solution has a structure in which spherical micelles and plate-like micelles are reversibly present. In the present invention, preferably, the concentration of the phospholipid is controlled to increase so that the plate-like micelles ate predominant.

FIG. 2B is a view showing the cross-sectional structure of a pre-emulsion base manufactured by adding saturated fatty alcohol having 12 to 22 carbon atoms to the hydrogenated lecithin dispersion solution at 70 to 90° C. with agitation to thus perform dispersion.

According to FIG. 2B, the saturated fatty alcohol having 12 to 22 carbon atoms healed to 70 to 90° C. is slowly added to the hydrogenated lecithin dispersion solution with agitation for 8 to 12 minutes (preferably for 10 minutes) under the condition of an ACl mixer (a general mixer) rotation speed of 800 to 1000 rpm, thus manufacturing a pre-emulsion base. The manufactured pre-emulsion base has a structure in which a saturated fatty alcohol is interposed between the hydrogenated lecithins of the plate-like micelles. As described above, the saturated fatty alcohol is interposed between the hydrogenated lecithins of the plate-like micelles, thereby stabilizing the unstable plate-like micelle structure.

FIG. 2C is a view showing the cross-sectional structure of a composition material base for skin moisturizing, manufactured by adding the pre-emulsion base and a water-soluble polypeptide, which is a water-soluble substance, to the purified water at 70 to 90° C. with agitation to thus perform dispersion.

According to FIG. 2C, the pre-emulsion base and RH-polypeptide-64 (aquaporin). which is a water-soluble polypeptide, heated to 70 to 90° C. are slowly added to the purified water with agitation for 8 to 12 minutes (preferably for 10 minutes) under the condition of an ACl mixer (a general mixer) rotation speed of 800 to 1000 rpm, thus manufacturing an emulsion base. In the manufactured emulsion base, the plate-like micelles are elongated by the water-soluble polypeptide and have a multi-layer structure. Since the plate-like micelles are elongated by the water-soluble polypeptide and have the multi-layer structure, preparation for easy formation of the multi-layer globule is completed. In some cases, a water-soluble natural moisturizing factor and a water-soluble moisturizing-membrane-forming agent may be added to the water-soluble polypeptide.

FIG. 2D is a view showing the cross-sectional structure of a multi-layer globule for skin moisturizing including phospholipid bilayers manufactured by adding oil to the composition material base for skin moisturizing with agitation to thus perform dispersion.

According to FIG. 2D, the oil heated to 70 to 90° C. is slowly added to the emulsion base at 70 to 90° C. with agitation for 8 to 12 minutes (preferably for 10 minutes) under conditions of a homo mixer (a mixer for up-and-down agitation) rotation speed of 3000 to 4000 rpm and a paddle mixer (a mixer for left-and-right agitation) rotation speed of 40 to 60 rpm. thus manufacturing a multi-layer globule including phospholipid bilayers. In the case of the manufactured multi-layer globule including the phospholipid bilayers, thee plate-like micelles which are elongated by the water-soluble polypeptide and which have the multi-layer structure wrap the oil and form a closed structure of a spherical globule, and both ends thereof are connected to each other to thus form a spherical vehicle having a multi-layer structure. A plurality of phospholipid bilayers (phospholipid membranes I, II, and III) forms closed structures of a spherical multi-layer globule for skin moisturizing. In some cases, an oil-soluble moisturizing-membrane-forming agent may be added to the oil.

The water-soluble polypeptide, the water-soluble natural moisturizing factor, or the water-soluble moisturizing-membrane-forming agent is located in a water-soluble phase portion between the phospholipid bilayers, and the oil or the oil-soluble moisturizing-membrane-forming agent is located in an oil-soluble phase portion in the phospholipid bilayer. Since the water-soluble polypeptide, the water-soluble natural moisturizing factor, or the moisturizing-membrane-forming agent are separated and are stably present for each layer of the multi-layer structure, the water-soluble polypeptide, the water-soluble natural moisturizing factor, or the moisturizing-membrane-forming agent may be stably delivered to the inside of the skin without destroying the vehicle.

When the temperature condition and the rotation speed conditions of the mixer, the homo mixer, and the paddle mixer specified in the present invention are not satisfied, the multi-layer globule for skin moisturizing including the phospholipid bilayers is not manufactured. Particularly, if the process conditions in which the oil heated to 70 to 90° C. is slowly added to the composition material base for skin moisturizing at 70 to 90° C. with agitation for 8 to 12 minutes (preferably for 10 minutes) under conditions of a homo mixer (a mixer for up-and-down agitation) rotation speed of 3000 to 4000 rpm and a paddle mixer (a mixer for left-and-right agitation) rotation speed of 40 to 60 rpm are not satisfied, the multi-layer structure for skin moisturizing may not be formed.

In the step of manufacturing the hydrogenated lecithin dispersion solution, after the hydrogenated lecithin is dissolved in ethanol, the hydrogenated lecithin that is dissolved in ethanol is added to the purified water at 55 to 70° C. with agitation to thus perform dispersion, thereby manufacturing the hydrogenated lecithin dispersion solution. Since the pre-treatment process of ethanol is performed, the temperature of the step of manufacturing the hydrogenated lecithin dispersion solution may be reduced from 70 to 90° C. to 55 to 70° C., thereby preventing the possibility that the hydrogenated lecithin is denatured at high temperatures.

In the step of manufacturing the pre-emulsion base by adding the saturated fatly alcohol having 12 to 22 carbon atoms to the hydrogenated lecithin dispersion solution at 70 to 90° C. with agitation to thus perform dispersion, one or mote among ceramide. sterol, or saturated fatty acid having 12 to 22 carbon atoms may be added to the saturated fatty alcohol. In this case, the saturated fatty alcohol having 12 to 22 carbon atoms must be reduced by the amount of the ceramide, the sterol, or the saturated fatty acid having 12 to 22 carbon atoms that is added. However, when the ceramide, the sterol, or the saturated fatty acid having 12 to 22 carbon atoms is added in an excessive amount, the unstable plate-like micelle structure is stabilized, but there is a problem in that the two ends thereof are connected to each other to form a spherical shape before the plate-like micelles form a multi-layer structure.

In the present invention, the pre-emulsion base may be manufactured using 30 to 75 parts by weight of the purified water, 10 to 30 parts by weight of the hydrogenated lecithin, and 15 to 40 parts by weight of the saturated fatty alcohol having 12 to 22 carbon atoms. When the composition of the pre-emulsion base is out of the above-described range, the saturated fatly alcohol is interposed between the hydrogenated lecithins of the plate-like micelles, and thus the effect of stabilizing the unstable plate-like micelle structure may not be obtained. As a result, the dermatologically active substance vehicle having the multi-layer structure may not be manufactured.

In the present invention, the composition material base for skin moisturizing may be manufactured using 30 to 75 parts by weight of the purified water, 15 to 40 parts by weight of the pre-emulsion base, and 10 to 30 pans by weight of the water-soluble polypeptide. When the composition of the composition material base for skin moisturizing is out of the above-mentioned range, the plate-like micelles which are elongated by the water-soluble poly peptide and which have the multi-layer structure are not formed, so that the multi-layer globule for skin moisturizing is not easily formed, and as a result, the composition material for skin moisturizing containing the vehicle having the multi-layer globule may not be manufactured.

In the present invention, the multi-layer globule for skin moisturizing including the phospholipid bilayers may be manufactured using 30 to 75 parts by weight of the composition material base for skin moisturizing and 5 to 25 parts by weight of the oil. When the composition of the multi-layer globule for skin moisturizing including the phospholipid bilayers is out of the above-mentioned range, the plate-like micelles having the multi-layer structure do not wrap the oil and do not form a closed structure of a spherical globule. As a result, the composition material for skin moisturizing having the spherical multi-layer globule few skin moisturizing may not be manufactured.

In the present invention, 15 to 40 parts by weight of the saturated fatty alcohol having 12 to 22 carbon atoms may include 10 to 30 parts by weight of saturated fatty alcohol having 12 to 20 carbon atoms and 5 to 10 parts by weight of behenyl alcohol. In this case, the reason for adding behenyl alcohol is to reduce the viscosity of the composition material for skin moisturizing containing the vehicle having the multi-layer globule having the multi-layer structure. When the amount of the behenyl alcohol is less than 5 parts by weight, the viscosity reduction effect is very small. When the amount of the behenyl alcohol is more than 10 parts by weight, the viscosity becomes very low.

In the present invention, the reason why saturated fatty alcohol having 12 to 22 carbon atoms is used as the above-described saturated fatty alcohol is that even though the viscosity of the emulsified product is increased when the number of carbon atoms constituting the saturated fatty alcohol is increased, the viscosity is reduced when the number of carbon atoms is 22 or more. Saturated fatty alcohol having 22 carbon atoms may be further mixed to control the viscosity of the emulsified product. Further, saturated fatty alcohol having fewer than 12 carbon atoms does not form a double-membrane (phospholipid bilayer) structure, which is the basic structure of the vehicle, due to the shore carbon chain thereof. Even when a double-membrane structure is formed, the stability of the formed multi-layer vehicle is poor.

In some cases, in order to solve the problem of an increase in the viscosity of the emulsified product as the number of carbon atoms constituting the saturated fatty alcohol increases, a saturated fatty acid having 12 to 22 carbon atoms may be further included. The reason why the added saturated fatty acid has 12 to 22 carbon atoms is that since a saturated fatty alcohol having 12 to 22 carbon atoms is used in the present invention, the compatibility therebetween can be enhanced.

### Mode for the Invention

1. Test Example 1: Pre-Treatment Process for Manufacturing Pre-Emulsion Base (Pre-Mixing Process for Manufacturing Multi-Layer Vehicle)

TABLE 1

| Phase | Raw material name | Wt % | Remarks |
|---|---|---|---|
| A | Water | 75.00~30.00 | Solvent |
| B | Hydrogenated lecithin | 10.00~30.00 | Emulsifier |
| C | Cetyl alcohol or Stearyl | 10.00~30.00 | Emulsion stabilizer |
| | alcohol orCetostearyl | | |
| | alcohol or | | |
| D | Behenyl alcohol | 5.00~10.00 | Emulsion stabilizer |
| | Total | 100.00 | |

A pre-emulsion base was prepared in the composition range shown in Table 2 using a pre-treatment process for manufacturing a dermatologically active substance vehicle having a multi-layer structure.

Specific conditions of the pre-treatment process for manufacturing the dermatologically active substance vehicle having the multi-layer structure are as follows.

Process 1: A phase A is put into a main kiln and heated to 80° C.

Process 2: While keeping the temperature at SOX, a phase B is slowly added to the phase A, agitated, and dissolved (Agitation condition: AGI mixer 900 rpm (or 800 to 1000 ) rpm), 10 to 20 min).

Process 3: A phase D is put into an auxiliary kiln containing the phase C and is dissolved while the temperature is increased to 80° C. (Agitation condition: AGI mixer 300 rpm).

Process 4: The phase AC is put into the phase A at 80° C. and agitated using the AGI mixer to thus perform mixing (Agitation condition: AGI mixer 900 rpm (or 800 to 1000 rpm), 10 to 20 min).

Process 5: The phases A, B, C, and D, mixed by agitation, are cooled to 50° C. and then stored at room temperature in the state of being sealed in a reservoir.

For reference, the constitution of the substance and the pre-treatment process may be modified and changed as described below according to the purpose of use in the skin, such as the maintenance of stability of the composition material for skin moisturizing containing the vehicle having the multi-layer globule, and skin moisturizing and protection. Accordingly, the following additional options are available in the pre-treatment process for manufacturing the pre-emulsion base (pre-mixing process for manufacturing the multi-layer vehicle).

Option 1: As in the step of manufacturing the saturated fatty alcohol dispersion solution of the present invention, a process of Firstly dissolving the phase B in ethanol having a purity of 95% or higher and then putting the resultant phase into the phase A may be added when the phase B is put into the phase A and dissolved therein.

Option 2: A ceramide raw material may be added to the phase C in Table 2, thereby performing the pre-treatment process. The saturated fatty alcohol must be removed in an amount that is in proportion to the content of added ceramides when mixing.

Option 3: A sterol raw material maybe added in the range of 0.10 to 2.00% to the phase C in Table 2. thereby performing the pre-treatment process. However, the saturated fatty alcohol must be removed in an amount that is in proportion to the content of added sterols when mixing.

Option 4: A predetermined amount of fatty acid may be added to the phase C in Table 2. thereby firmly strengthening the phospholipid membrane including the phospholipid bilayers of the dermatologically active substance vehicle having the multi-layer structure or lowering the viscosity of a composition material for the finally manufactured functional cosmetic. The type of fatty acid that can be used is saturated fatty acid having 12 to 22 carbon atoms. Among the acids, long-chain fatty acid and very-long-chain fatty acid are generally used. Among them, behenic acid is particularly preferable. Behenic acid comparatively more strongly affects the viscosity of the composition material for the finally manufactured functional cosmetic than other long-chain fatty acids or very-long-chain fatty acids. Accordingly, the content thereof must be carefully controlled. However, the saturated fatty alcohol must be removed in an amount that is in proportion to the content of added fatty acid when mixing.

In Table 2, the constitution of the substance includes 1) purified water, 2) hydrogenated lecithin, and 3) saturated fatty alcohol. The saturated fatty alcohol acts as an emulsion stabilizer and is used in order to lower the high interfacial tension of the hydrogenated lecithin, and the hydrogenated lecithin having both hydrophilic and hydrophobic properties acts as an emulsifier. In addition, the saturated fatty alcohol that is used may be used alone or in a combination of two or more thereof, and saturated fatty alcohol having 12 to 22 carbon a oms is used as a main ingredient In order to lower the viscosity of a product obtained by emulsification, C22 behenyl alcohol may be further used in combination therewith.

In order to positively affect the physical properties of the dermatologically active substance vehicle, the state of emulsion panicles, and the manufacturing process when the composition material for the pre-emulsion base is prepared, 1) ceramide, 2) sterol, 3) fatty acid, and 4) ethanol may be added to modify the composition material. For example, in order to strengthen the phospholipid bilayers (phospholipid membranes I, II, and III) of the multi-layer structure emulsion panicle, one or more of ceramide. sterol, or saturated fatty acid having 12 to 22 carbon atoms may be added during the step of manufacturing the pre-emulsion base by adding the saturated fatty alcohol having 12 to 22 carbon atoms to the hydrogenated lecithin dispersion solution at 70 to 90° C. with agitation to thus perform dispersion. Since the hydrogenated lecithin has a hydrophilic head and a hydrophobic tail and is not high in terms of solubility in water at room temperature, after the hydrogenated lecithin is first dissolved in ethanol, the hydrogenated lecithin that is dissolved in ethanol may be added to purified water heated to 55 to 70° C. with agitation to thus perform dispersion.

Further, care should be taken to control the temperature in the pre-treatment process (pre-mixing process) for manufacturing the composition material for skin moisturizing containing the vehicle having the multi-layer globule. When the temperature is maintained at 70 to 90° C. over a long period of time, the hydrogenated lecithin is hardened due to the temperature effect, and the function of the hydrogenated lecithin is remarkably weakened. As a result, the ability to generate a vesicle, which is a closed endoplasmic reticulum, that is, a liposome, is lowered, and problems occur in the moisturizing performance of the composition material for skin moisturizing containing the vehicle having the multi-layer globule due to the decrease in the stability of the dermatologically active substance vehicle that is generated. Therefore, it is recommended to cool the pre-emulsion base produced using the pre-treatment process (pre-mixing process) for manufacturing the dermatologically active substance vehicle as fast as possible and to store the cooled pre-emulsion base at room temperature.

In addition, the hydrogenated lecithin has a hydrophilic head and a hydrophobic tail, and is not high in terms of solubility is water at room temperature. In this regard, there are 1) a method of manufacturing a pre-emulsion base by adding saturated fatty alcohol having 12 to 22 carbon atoms to a hydrogenated lecithin dispersion solution at 70 to 90° C. with agitation to thus perform dispersion, and 2) a method of dissolving hydrogenated lecithin in ethanol and then adding the hydrogenated lecithin dissolved in ethanol to purified water heated to 55 to 70° C. with agitation to thus perform dispersion. As a result, it is possible u disperse the hydrogenated lecithin in water using the above two methods, and there is no difference in the degree of dispersion or physical properties of the two methods regardless of which method is selected because the selection relates to shortening of the dispersion process time and the efficiency. Ceramide and sterol are a kind of lipid membrane ingredients of the epidermal stratum corneum among skin cells. They are substances that prevent water loss from the skin surface and block the infiltration of harmful substances from the outside.

Ceramide plays a role as a barrier to lipid layers by structurally bonding with water, and has functions of protecting the body from the external environment and microorganisms and of regulating cell growth as a signaling system in terms of physiology. The role of ceramide and sterol in the process of manufacturing the dermatologically active substance vehicle having the multi layer structure is preferably to arrange hydrogenated lecithin particles and to be provided between the hydrogenated lecithin particles having a double-membrane structure including the phospholipid bilayers so that the membrane of the double-membrane structure is made more dense, thus improving the stability of the dermatologically active substance vehicle having the multi-layer structure. As shown in Table 2, finally, the pre-emulsion base obtained during the pre-treatment process for manufacturing the dermatologically active substance vehicle having the multi-layer structure is preferably used in a content in the range of 10.00 to 20.00 wt % in the functional cosmetic including the composition material for skin moisturizing containing the vehicle having the multi-layer globule.

2. Test Example 2: Composition Material for Skin Moisturizing Containing a Vehicle Having a Multi-Layer Globule Including Phospholipid Bilayers Manufactured Using Pre-Emulsion Base (Manufacture of Skin Multi-Layer Vehicle for Moisturizing Using Pre-Mixing Process)

TABLE 2

| Phase | Raw material name | Wt %/% | Remarks |
|---|---|---|---|
| A | Green tea water | 44.86 | |
| | Pre-emulsion base | 16.00 | |
| | Xanthan gum, glycerin, panthenol | 6.05 | Natural moisturizing factor |
| | (D form) | | |
| | Shea butter glycerides, sodium olivoyl | 2.00 | Natural moisturizing factor and |
| | glutamate | | moisturizing-membrane-forming agent |
| | RH-polypeptide-64 (Aquaporin) | 2.00 | Water-soluble polypeptide |
| | VENNARC-001 BIOACTIVES | 10.00 | |
| | Eoseongcho extract | 2.00 | |
| | Disodium EDTA, arginine (L type) | 0.15 | |
| | Dipotassium glycyrrhizate, adenosine | 0.24 | |
| | Nylon-12 | 1.00 | |
| B | Cetostearyl alcohol | 2.00 | |
| | Stearic acid | 0.80 | |
| | Sunflower seed oil, Corylus avellana | 6.00 | |
| | seed oil | | |
| | Cocoglycerides, Lupinus albus oil | 2.50 | |
| | Shea butter, beeswax | 0.80 | Moisturizing-membrane-forming |
| | | | agent |
| | Glyceryl stearate, cetearyl glucoside | 1.50 | |
| C | Carbomer | 0.10 | |
| D | Gold extract, peony root extract | 2.00 | |
| | Total | 100.00 | |

[Process of Manufacturing Composition Material for Skin Moisturizing Containing a Multi-Layer Globule for Skin Moisturizing]

Process 1. A phase A is put into a main kiln and then heated to BOX with agitation to thus perform dissolving (Agitation conditions: Paddle mixer at 50 rpm).

Process 2. The phase A is dissolved while being heated to 80° C., and is then uniformly dispersed using a paddle mixer and a homo mixer (Agitation conditions: Paddle mixer 50 rpm. Homo mixer 3000 rpm. 10 min at 80° C.).

Process 3. A phase B is put into an auxiliary kiln and is then dissolved while being heated to 80° C. (Agitation conditions: Dispersing mixer 200 rpm).

Process 4. The phase B is put into the phase A, and is then emulsified with agitation under a constant temperature condition of BOX (Agitation conditions: Paddle mixer 50 rpm. Homo mixer 3000 rpm. 8 min, at 80° C.).

Process 5. The contents contained in the main kiln are cooled to 45° C. with deaeration (Agitation conditions: Paddle mixer 40 rpm, Cooling up to 40° C. under vacuum).

Process 6. The phase C is put into the main kiln and the contents arc then uniformly agitated (Agitation conditions: Paddle mixer 40 rpm. Homo mixer 2200 rpm, 4 min).

Process 7. The phase D is put into the main kiln and the contents arc then uniformly agitated (Agitation conditions: Paddle mixer 40 rpm. Homo mixer 2200 rpm. 4 min).

Process 8. The contents contained in the main kiln are subjected to second deaeration while being cooled to 35° C. (Agitation conditions: Paddle mixer 40 rpm. Cooling up to 35° C. under vacuum).

Process 9. The contents contained in the main kiln are floated in a separate reservoir and then aged at room temperature for 3 days.

Particularly, when in process 2, uniform dispersion is not performed at a temperature of 80° C. for 10 min under conditions of a homo mixer (a mixer for up-and-down agitation) rotation speed of 3000 rpm and a paddle mixer (a mixer for left-and-right agitation) rotation speed of 50 rpm, or when in process 4, uniform dispersion is not performed at a temperature of 80° C. for 8 min under conditions of a homo mixer (a mixer for up-and-down agitation) rotation speed of 3000 rpm and a paddle mixer (a mixer for left-and-right agitation) rotation speed of 50 rpm, the composition material for skin moisturizing containing the vehicle having the multi-layer globule including the phospholipid bilayers may not be formed.

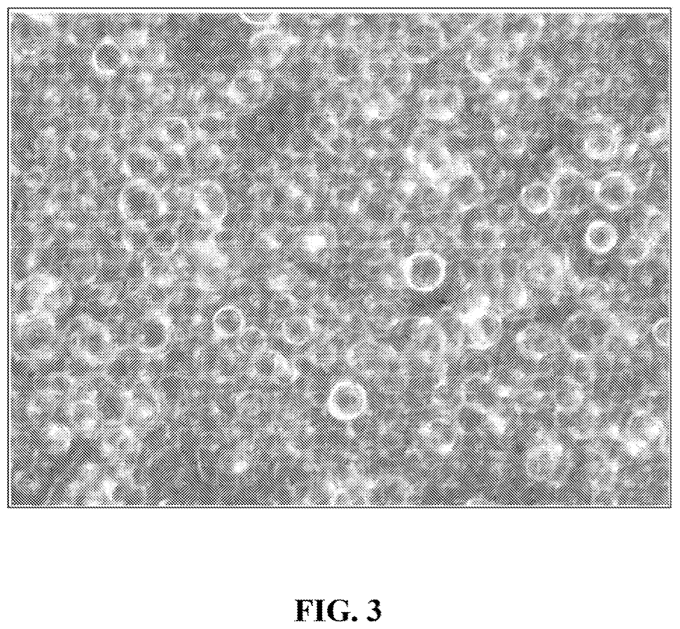

FIG. 3 is a picture of a composition material for skin moisturizing containing the vehicle having the multi-layer globule manufactured under the above-described process conditions, which is the present invention, taken at a magnification of 400 times using a polarizing microscope. From FIG. 3, it can be confirmed that the composition material for skin moisturizing containing the vehicle having the multi-layer globule which is the present invention is a multi-layer globule for skin moisturizing having a multi-layer structure including a plurality of phospholipid bilayers.

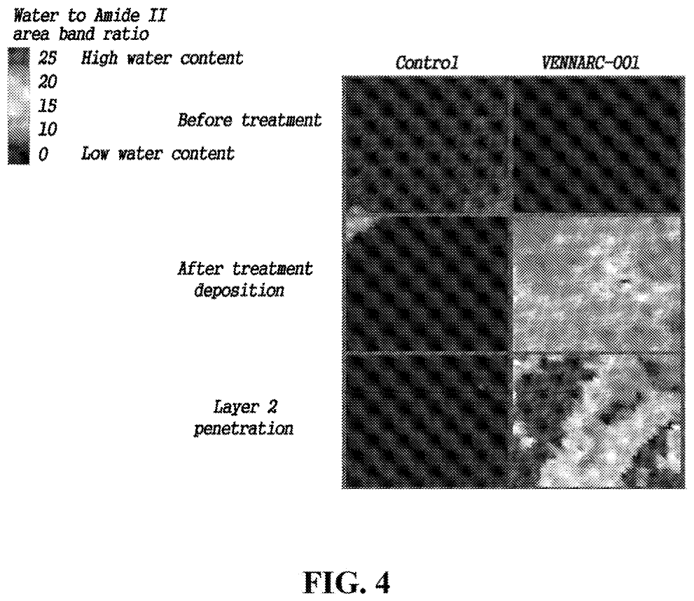

FIG. 4 is an ATR-FTIR image showing the hydration levels of the skin (control), which is not treated with the composition material for skin moisturizing containing the vehicle having the multi-layer globule that is the present invention, and the skin (VENNARC-001), which is treated with the composition material for skin moisturizing containing the vehicle having the multi-layer globule, using a peak area ratio of water to amide II cm⁻¹.

The hydration levels of the skin not heated and treated with the composition material for skin moisturizing containing the vehicle having the multi-layer globule manufactured using the composition and the process of Table 2 were compared and measured. In the ATR-FTIR image showing the hydration levels of the skin (control), which is not treated with the composition material for skin moisturizing containing the vehicle having the multi-layer globule, and the skin (VENNARC-001), which is treated with the composition material for skin moisturizing containing the vehicle having the multi-layer globule, using a peak area ratio of water to amide II cm⁻¹, an increase in the red color means an increased hydration level. Based on this comparison, FIG. 4 shows that the hydration level of the inside of the stratum corneum is high in the skin (VENNARC-001) treated win the composition material for skin moisturizing. That is, FIG. 4 shows that the hydration level is higher in the skin treated with the composition material for skin moisturizing (VENNARC-001) than in the skin not treated with the composition material for skin moisturizing (Control).

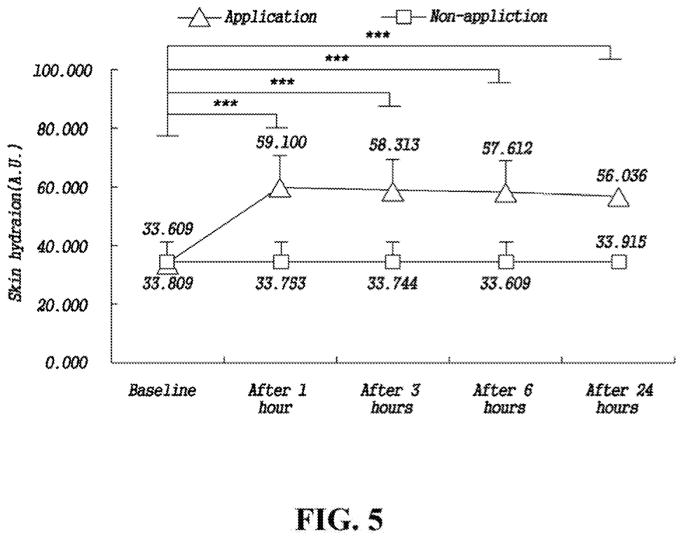

FIG. 5 is a comparative graph showing the hydration levels of the skin (non-application), which is not treated with the composition material for skin moisturizing containing the vehicle having the multi-layer globule that is the present invention, and the skin (application), which is treated with the composition material for skin moisturizing containing the vehicle having the multi-layer globule, over time.

This is for measuring whether the internal water supply system [internal water supply system by aquaporin], the method of supplying water to the skin from the outside [external water supply method by NMF], and 3) the prevention of water loss from the skin [protection against water loss from skin surface] are conceitedly and smoothly performed. The long-term skin hydration ability in an antebrachium was repeatedly measured using a corneometer and analysis using ANOVA was performed over the skin (non-application) not treated with the composition material for skin moisturizing containing the vehicle having the multi-layer globule and the skin (application) treated with the composition material for skin moisturizing containing the vehicle having the multi-layer globule. The result of measurement of the hydration level in the skin (application) 1 hour, 3 hours, 6 hours, and 24 hours after treatment with the composition material for skin moisturizing containing the vehicle having the multi-layer globule shows a hydration level statistically significantly higher than those of the skin (non-application) not treated with the composition material for skin moisturizing containing the vehicle having the multi-layer globule and a baseline (p<0.001).

From the above experimental results it can be seen that in the case of the skin (application) treated with the composition material for skin moisturizing containing the vehicle having the multi-layer globule, the composition material for skin moisturizing which is the present invention provides a sufficient hydration effect over a long period of time in the epidermal layer underneath the stratum corneum. Accordingly, it can be seen that the skin is continuously and actively hydrated by supplying the aquaporin to the stratum granulosum of the epidermis in the present invention.

Hereinafter, the amounts of water loss of the skin (non-application) not treated with the composition material for skin moisturizing containing the vehicle having the multi-layer globule and the skin (application) treated with the composition material for skin moisturizing containing the vehicle having the multi-layer globule were compared over samples of 50 persons.

[Expert mental Example] Comparison of amounts of water lost from skin by measurement of Vapometer (TEWL) value [Protection against water loss from skin surface]

TABLE 3

| | | | | 25% (Loss | 75% (Loss |
|---|---|---|---|---|---|
| | N | Missing | Median | average value | average value |
| | (Sample | (Sample | (Median | up to the | to the |
| Group | number) | loss) | value) | top 25%) | bottom 75%) |
| Baseline | 50 | 0.000 | 13.850 | 11.475 | 17.925 |
| (No composition | | | | | |
| material treatment) | | | | | |
| 1st week | 50 | 0.000 | 14.700 | 11.925 | 19.650 |
| 2nd week | 50 | 0.000 | 14.700 | 11.250 | 18.250 |
| 4th week | 50 | 0.000 | 12.150 | 10.300 | 16.600 |
| 6th week | 50 | 0.000 | 12.600 | 10.975 | 15.150 |

Table 3 shows the results obtained by repeatedly measuring and analyzing the amount of water loss of the baseline not treated with the composition material for skin moisturizing containing the multi-layer globule for skin moisturizing including live phospholipid bilayers, and the amount of water loss at the 1st week, 2nd week, 4th week, and 6th week after treatment with the composition material for skin moisturizing containing the multi-layer globule for skin moisturizing using a Friedman's method (Friedman repeated measures analysis of variance on ranks, P=<0.01).

TABLE 4

| Baseline vs. | P | Significance |
|---|---|---|
| 1st week | 0.313 | No |
| 2nd week | 0.521 | No |
| 4th week | 0.035 | No |
| 6th week | 0.005 | Yes |

Table 4 shows comparative results of the amount of water loss of the baseline (control group) not treated with the composition material for skin moisturizing containing the multi-layer globule for skin moisturizing including the phospholipid bilayers and the amount of water loss of the group (treatment group) treated with the composition material for skin moisturizing containing the multi-layer globule for skin moisturizing (Multiple comparisons: Wilcoxen signed-rank test w/Bonferroni-adjusted p-value of 0.013(0.050/4)).

In general, the evaporation rate of water is measured using a vapometer. The higher the evaporation rate of water, the greater the loss of water between the epidermis. A lower evaporation rate means provision of better water-membrane and moisturizing functions of the epidermis to prevent water loss. From a comparison with the baseline (control group), it can be seen that in the case of the group (treatment group) treated with the composition material for skin moisturizing, the amount of water loss (TEWL) of the epidermis gradually decreases with the lapse of time and statistically significantly decrease at the 6th week. In general, a p value of 0.01 or less means that the group (treatment group) treated with the composition material for skin moisturizing has a statistically significant effect of preventing water loss compared to the baseline (control group) not treated with the composition material for skin moisturizing. It could be confirmed that the group (treatment group) treated with the composition material for skin moisturizing has a p value of 0.005 at the 6th week, which means a statistically significant reduction in the amount of water loss (TEWL) of the epidermis, compared to the baseline (control group). That is, it could be confirmed that in the case of treatment with the composition material for skin moisturizing containing the vehicle having the multi-layer globule which is the present invention, a moisturizing effect is excellent.

Hereinafter, compositions of skin cream, essence, a mask pack solution, and body cream are shown as examples of cosmetic products manufactured using the composition material for skin moisturizing containing the vehicle having the multi-layer globule which is the present invention.

TABLE 5

| Phase | Raw material name | Wt %/% | Remarks |
|---|---|---|---|
| A | Purified water | 40.06 | |
| | Pre-emulsion base | 18.00 | |
| | Sodium hyaluronate (1% solution) | 3.00 | Natural moisturizing factor |
| | Xanthan gum | 5.00 | Moisturizing-membrane-forming agent |
| | RH-polypeptide-64 | 3.00 | Water-soluble polypeptide |
| | Disodium EDTA | 0.04 | |
| | Allantoin | 0.10 | |
| | Arginine | 0.20 | |
| | Glycerin | 10.00 | Natural moisturizing factor |
| B | Cetyl alcohol | 2.00 | |
| | Stearic acid | 0.80 | |
| | Jojoba oil, Sunflower seed oil | 12.00 | |
| | Microcrystalline wax | 0.80 | Moisturizing-membrane-forming agent |
| | Jojoba butter | 1.00 | Moisturizing-membrane-forming agent |
| | Dimethicone | 0.50 | Moisturizing-membrane-forming agent |
| | Tocopheryl acetate | 0.20 | |
| | Glyceryl stearate | 1.00 | |
| C | Carbomer | 0.20 | |
| D | Flavoring | 0.10 | |
| E | 1,2-hexanediol | 2.00 | |

Table 5 shows the composition of skin cream which is a cosmetic product manufactured using a composition material for skin moisturizing containing a multi-layer globule.

TABLE 6

| Phase | Raw Materials Name | Wt %/% | Remarks |
|---|---|---|---|
| A | Purified water | 48.26 | |
| | Pre-emulsion base | 15.00 | |
| | Betaine | 3.00 | Natural moisturizing factor |
| | Xanthan gum | 5.00 | Moisturizing-membrane-forming agent |
| | RH-polypeptide-64 | 4.00 | Water-soluble polypeptide |
| | Disodium EDTA | 0.04 | |
| | Allantoin | 0.10 | |
| | Arginine | 0.10 | |
| | Glycosaminoglycans | 10.00 | Natural moisturizing factor |
| B | Cetostearyl alcohol | 1.00 | |
| | Stearic acid | 0.50 | |
| | Cocoglycerides, Sunflower seed oil | 7.00 | |
| | Beeswax | 0.80 | Moisturizing-membrane-forming agent |
| | Dimethicone | 1.00 | Moisturizing-membrane-forming agent |
| | Tocopheryl acetate | 0.50 | |
| | Silicone gum | 1.00 | Moisturizing-membrane-forming agent |
| | Glyceryl stearate, PEG-100 stearate | 1.50 | |
| C | Carbomer | 0.10 | |
| D | Flavoring | 0.10 | |
| E | 1,2-hexanediol | 2.00 | |

Table 6 shows the composition of essence which is a cosmetic product manufactured using a composition material for skin moisturizing containing a multi-layer globule.

TABLE 7

| Phase | Raw Materials Name | Wt %/% | Remarks |
|---|---|---|---|
| A | Purified water | 64.79 | |
| | Pre-emulsion base | 10.00 | |
| | Hydrolyzed collagen | 3.00 | Natural moisturizing factor |
| | Xanthan gum | 5.00 | Moisturizing-membrane-forming agent |
| | RH-polypeptide-64 | 1.00 | Water-soluble polypeptide |
| | Disodium EDTA | 0.04 | |
| | Allantoin | 0.10 | |
| | Arginine | 0.06 | |
| | Glycerin | 5.00 | Natural moisturizing factor |
| B | Cetostearyl alcohol | 0.70 | |
| | Stearic acid | 0.30 | |
| | Cocoglycerides, Sunflower seed oil | 4.00 | |
| | Mango seed butter | 0.10 | Moisturizing-membrane-forming agent |
| | Dimethicone | 0.50 | Moisturizing-membrane-forming agent |
| | Tocopheryl acetate | 0.10 | |
| | Glyceryl stearate, PEG-100 stearate | 1.00 | |
| C | Carbomer | 0.06 | |
| D | Flavoring | 0.05 | |
| E | 1,2-hexanediol | 2.00 | |

Table 7 shows the composition of a mask pack solution which is a cosmetic product manufactured using a composition material for skin moisturizing containing a multi-layer globule.

TABLE 8

| Phase | Raw Materials Name | Wt %/% | Remarks |
|---|---|---|---|
| A | Purified water | 52.31 | |
| | Pre-emulsion base | 12.00 | |
| | Glycerin | 3.00 | Natural moisturizing factor |
| | Xanthan gum | 5.00 | Moisturizing-membrane-forming agent |
| | RH-polypeptide-64 | 2.00 | Water-soluble polypeptide |
| | Disodium EDTA | 0.04 | |
| | Allantoin | 0.10 | |
| | Arginine | 0.10 | |
| | Hydrolyzed collagen | 2.00 | Natural moisturizing factor |
| | Sodium olivoyl glutamate | 2.00 | Moisturizing-membrane-forming agent |
| B | Cetostearyl alcohol | 1.50 | |
| | Stearic acid | 0.60 | |
| | Olive oil, Sunflower seed oil | 10.00 | |
| | Shea butter | 3.00 | Moisturizing-membrane-forming agent |
| | Dimethicone | 0.50 | Moisturizing-membrane-forming agent |
| | Tocopheryl acetate | 0.20 | |
| | Glyceryl stearate, PEG-100 stearate | 1.50 | |
| C | Carbomer | 0.10 | |
| D | Flavoring | 0.05 | |
| E | 1,2-hexanediol | 2.00 | |

Table 8 shows the composition of body lotion which is a cosmetic product manufactured using a composition material for skin moisturizing containing a multi-layer globule.

While the present invention has been described with reference to exemplary embodiments thereof, it is to be understood that the present invention is not limited to the disclosed exemplary embodiments, but on the contrary, those skilled in the art will appreciate that various amendments and modifications are possible front the description. Accordingly, it is intended that the idea of the present invention be defined only by the claims appended hereto, and that all equivalents or equivalent variations thereof fall within the scope of the present invention.

1. A method of manufacturing a composition material for skin moisturizing containing a vehicle having a multi-layer globule, the method comprising:
manufacturing a hydrogenated lecithin dispersion solution by adding hydrogenated lecithin to purified water at 70 to 90° C. with agitation to thus perform dispersion;
manufacturing a pre-emulsion base by adding saturated fatty alcohol having 12 to 22 carbon atoms to the hydrogenated lecithin dispersion solution at 70 to 90° C. with agitation to thus perform dispersion;
manufacturing a composition material base for skin moisturizing by adding the pre-emulsion base and water-soluble polypeptide to the purified water at 70 to 90° C. with agitation to thus perform dispersion; and
manufacturing a multi-layer globule for skin moisturizing including phospholipid bilayers by adding oil to the composition material base for skin moisturizing at 70 to 90° C. with agitation to thus perform dispersion.

2. The method of claim 1, wherein the water-soluble polypeptide is RH-polypeptide-64 (aquaporin).

3. The method of claim 1, wherein a natural moisturizing factor and a water-soluble moisturizing-membrane-forming agent are added to the water-soluble polypeptide.

4. The method of claim 1, wherein an oil-soluble moisturizing-membrane-forming agent is added to the oil.

5. The method of claim 3, wherein the natural moisturizing factor is one or more among glycerin, panthenol (D form), betaine, sodium hyaluronate, amino acid, collagen, glycosaminoglycan, sodium chondroitin sulfate, or mucopolysaccharide.

6. The method of claim 4, wherein the water-soluble moisturizing-membrane-forming agent is one or more among glyceride, sodium olivoyl glutamate, or xanthan gum, and the oil-soluble moisturizing-membrane-forming agent is one or more among shea butter, beeswax, vegetable wax, mineral wax, animal wax, silicone, silicone gums, silicone emulsions, silicone elastomers, or vegetable butter.

7. The method of claim 1, wherein the manufacturing the multi-layer globule for skin moisturizing including the phospholipid bilayers is performed for 8 to 12 minutes under conditions of a homo mixer rotation speed of 3000 to 4000 rpm and a paddle mixer rotation speed of 40 to 60 rpm.

8. The method of claim 7, wherein the manufacturing the hydrogenated lecithin dispersion solution is performed for 8 to 12 minutes under a condition of a mixer rotation speed of 800 to 1000 rpm, the manufacturing the pre-emulsion base is performed for 8 to 12 minutes under a condition of a mixer rotation speed of 800 to 1000 rpm, and the manufacturing the composition material base for skin moisturizing is performed for 8 to 12 minutes under a condition of a mixer rotation speed of 800 to 1000 rpm.

9. The method of claim 1, wherein the manufacturing the hydrogenated lecithin dispersion solution includes dissolving the hydrogenated lecithin in ethanol and then adding the hydrogenated lecithin dissolved in the ethanol to the purified water at 55 to 70° C. with agitation to thus perform dispersion, thereby manufacturing the hydrogenated lecithin dispersion solution.

10. The method of claim 1, wherein one or more among ceramide, sterol, or saturated fatty acid having 12 to 22 carbon atoms are added to the saturated fatty alcohol.

11. The method of claim 1, wherein the composition material for skin moisturizing has one type of formulation selected from the group consisting of solutions, suspensions, emulsions, gels, lotion, essence, cream, powder, soaps, packs, masks, cleansing, oil, foundations, or sprays.
